(19) United States
(12) Patent Application Publication
(10) Pub. No.: US 2022/0273393 A1
(43) Pub. Date: Sep. 1, 2022

(54) APPARATUS AND METHOD FOR PROVIDING SURGICAL ENVIRONMENT BASED ON A VIRTUAL REALITY

(71) Applicant: HUTOM Co., Ltd., Seoul (KR)

(72) Inventors: Jihun YOON, Paju-si (KR); Seul Gi HONG, Incheon (KR); Seung Bum HONG, Seoul (KR); Sung Hyun PARK, Seoul (KR); Min Kook CHOI, Seoul (KR)

(73) Assignee: HUTOM Co., Ltd., Seoul (KR)

(21) Appl. No.: 17/525,524

(22) Filed: Nov. 12, 2021

Related U.S. Application Data
(63) Continuation of application No. PCT/KR2021/014478, filed on Oct. 18, 2021.

(30) Foreign Application Priority Data
Feb. 26, 2021 (KR) 10-2021-0026519

(51) Int. Cl. A61B 90/00; G06T 7/246; G06F 3/01

(52) U.S. Cl. CPC A61B 90/37; A61B 90/361; G06T 7/251; G06F 3/011; A61B 2090/365; G06T 2207/10021; G06T 2207/10028; G06T 2207/30004; G06T 2207/20084

(57) ABSTRACT

The inventive concept relates to a method of providing surgical environment based on a virtual reality, and more particularly, to an apparatus and method for providing surgical environment based on a virtual reality by figuring out the movement of a surgical tool in a surgical video. According to the inventive concept, it is possible to generate a virtual surgical tool identical to an actual surgical tool in an actual surgical video based on virtual reality and determine the movement of the actual surgical tool according to the log information of the virtual surgical tool, thereby accurately identifying the movement of the actual surgical tool.

### CROSS-REFERENCE TO RELATED APPLICATIONS

The present application is a continuation of International Patent Application No. PCT/KR2021/014478, filed on Oct. 18, 2021, which is based upon and claims the benefit of priority to Korean Patent Application No. 10-2021-0026519 filed on Feb. 26, 2021. The disclosures of the above-listed applications are hereby incorporated by reference herein in their entirety.

### BACKGROUND

The inventive concept relates to a method of providing surgical environment based on a virtual reality, and more particularly, to an apparatus and method for providing surgical environment based on a virtual reality by figuring out the movement of a surgical tool in a surgical video.

Recently, in the case of surgery in a hospital, conditions of a patient before surgery may be implemented into a three-dimensional simulation (stereoscopic video) without immediately proceeding with the surgery and the surgery may be performed virtually under the same conditions as the actual surgery.

More specifically, in the case of virtual simulation surgery, a precise diagnosis may be established in advance. Therefore, it is possible to make a plan through virtual simulation surgery rather than relying on the sense of a specialist, and to reduce even the minor error.

The surprising effect of the virtual simulation surgery is that the accuracy of the surgery is improved, the actual surgery situation is predicted, and the surgical method suitable for each patient is provided, thereby reducing the time.

However, it is very important to set a basic environment for performing the virtual simulation surgery to increase the accuracy of the virtual simulation surgery. Specifically, data on virtual surgical tools, which are essential when performing virtual simulation surgery, are required.

### SUMMARY

Embodiments of the inventive concept provide to an apparatus and method for generating a virtual surgical tool identical to an actual surgical tool in an actual surgical video based on virtual reality and determining the movement of the actual surgical tool according to log information of the virtual surgical tool.

However, problems to be solved by the inventive concept may not be limited to the above-described problems. Although not described herein, other problems to be solved by the inventive concept can be clearly understood by those skilled in the art from the following description.

According to an embodiment, a method of providing surgical environment based on a virtual reality in an apparatus includes a) recognizing at least one actual surgical tool included in an actual surgical video for each preset frame in the actual surgical video based on a first artificial intelligence model, b) performing correspondence matching on at least one identical portion of the actual surgical tool and at least one virtual reality-based virtual surgical tool corresponding to the actual surgical tool, c) performing calibration such that the virtual surgical tool corresponds to a position of the actual surgical tool according to a result of the correspondence matching, d) calculating calibrated coordinate values of the virtual surgical tool, e) calculating a plurality of coordinate values for a positions which the virtual surgical tool is calibrated for each preset frame by repeatedly performing steps a) to d), and t) generating a plurality of pieces of log information based on a difference between the calculated plurality of coordinate values.

The performing of the correspondence matching may include setting at least one region of the actual surgical tool as a first reference point, setting a region identical to the at least one region of the actual surgical tool in the virtual surgical tool as a second reference point by using a semantic correspondence matching technique, and performing the correspondence matching using the first and second reference points.

The generating of the log information may include sequentially generating pieces of log information about a difference between a previously-calculated coordinate value and a subsequently-calculated coordinate value whenever the plurality of coordinate values are calculated.

The method may further include accumulating and storing the sequentially generated pieces of log information.

The accumulating and storing of the sequentially generated pieces of log information may include accumulating and storing currently generated log information only when a difference between the sequentially generated pieces of log information is equal to or greater than a preset difference.

The method may further include predicting a movement of the actual surgical tool changed from a current frame to a next frame in the actual surgical video based on the accumulated and stored log information, and displaying a visual effect representing the predicted movement at a position corresponding to the movement on the current frame in the actual surgical video.

The actual surgical video may be taken through a stereoscopic camera and includes a three-dimensional depth value for an actual surgical object for each frame.

The performing of the calibration may include rendering the virtual surgical tool as a three-dimensional object by assigning a corresponding three-dimensional depth value to the calibrated position of the virtual surgical tool.

The calculating of the coordinate values may include including the three-dimensional depth value in coordinate values corresponding to the calibrated position of the virtual surgical tool after the three-dimensional depth value is assigned.

The generating of the log information may include generating a plurality of pieces of log information to which the three-dimensional depth value is assigned based on a difference between a plurality of coordinate values calculated by including the three-dimensional depth value.

According to an embodiment, an apparatus for providing surgical environment based on a virtual reality includes a video acquisition unit configured to acquire an actual surgical video, a memory, and a processor that performs a first process of recognizing at least one actual surgical tool included in an actual surgical video for each preset frame in the actual surgical video based on a first artificial intelligence model, performs a second process of performing correspondence matching on at least one identical portion of the actual surgical tool and at least one virtual reality-based virtual surgical tool corresponding to the actual surgical tool, performs a third process of performing calibration such that the virtual surgical tool corresponds to a position of the actual surgical tool according to a result of the correspondence matching, performs a fourth process of calculating calibrated coordinate values of the virtual surgical tool, performs a fifth process of calculating a plurality of coordinate values for a positions which the virtual surgical tool is calibrated for each preset frame by repeatedly performing the first to fourth process, and performs a sixth process of generating a plurality of pieces of log information based on a difference between the calculated plurality of coordinate values.

In addition, another method for implementing the inventive concept, another system, and a computer-readable recording medium for recording a computer program for executing the method may be further provided.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a view for describing an apparatus for providing surgical environment based on a virtual reality according to the inventive concept.

FIG. 2 is a view for describing that a processor of the apparatus 10 according to the inventive concept calculates coordinate values of a virtual reality-based virtual surgical tool through actual surgical videos.

FIG. 3 is a view for describing that a processor of the apparatus according to the inventive concept generates log information of a virtual reality-based virtual surgical tool through actual surgical videos.

FIG. 4 is a view for describing that a processor of the apparatus according to the inventive concept calculates coordinate values of a virtual reality-based virtual surgical tool through actual surgical videos to which depth values are applied.

FIG. 5 is a view for describing that a processor of the apparatus according to the inventive concept generates log information of a virtual reality-based virtual surgical tool through actual surgical videos to which depth values are applied.

FIGS. 6A to 6C are diagrams for describing that a processor of the apparatus according to the inventive concept displays a visual effect indicating a predicted movement on an actual surgical video.

FIG. 7 is a flowchart illustrating a process in which a processor of the apparatus according to the inventive concept provides a virtual reality-based surgical environment.

FIG. 8 is a flowchart illustrating a process in which a processor of the apparatus according to the inventive concept performs correspondence matching.

### DETAILED DESCRIPTION

Advantages and features of the inventive concept and methods for achieving them will be apparent with reference to embodiments described below in detail in conjunction with the accompanying drawings. However, the inventive concept is not limited to the embodiments disclosed below, but can be implemented in various forms, and these embodiments are to make the disclosure of the inventive concept complete, and are provided so that this disclosure will be thorough and complete and will fully convey the scope of the invention to those of ordinary skill in the art, which is to be defined only by the scope of the claims.

The terminology used herein is for the purpose of describing particular embodiments only and is not intended to be limiting of the inventive concept. The singular expressions include plural expressions unless the context clearly dictates otherwise. In this specification, the terms “comprises” and/or “comprising” are intended to specify the presence of stated elements, but do not preclude the presence or addition of elements. Like reference numerals refer to like elements throughout the specification, and “and/or” includes each and all combinations of one or more of the mentioned elements. Although “first”, “second”, and the like are used to describe various components, these components are of course not limited by these terms. These terms are only used to distinguish one component from another. Thus, a first element discussed below could be termed a second element without departing from the teachings of the inventive concept.

Unless otherwise defined, all terms (including technical and scientific terms) used herein have the same meaning as commonly understood by one of ordinary skill in the art to which this invention belongs. Further, unless explicitly defined to the contrary, the terms defined in a generally-used dictionary are not ideally or excessively interpreted.

Hereinafter, the inventive concept will be described with reference to the accompanying drawings.

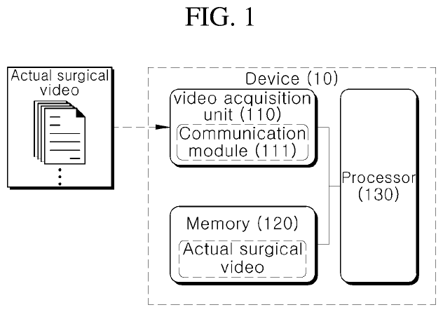

FIG. 1 is a diagram for describing an apparatus 10 for providing surgical environment based on a virtual reality according to the inventive concept.

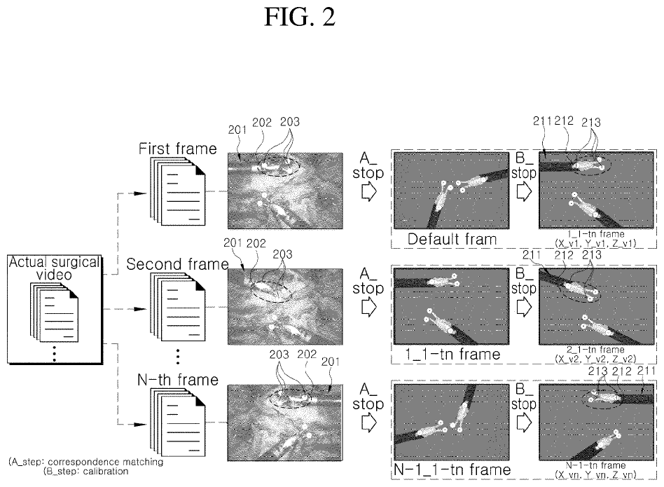

FIG. 2 is a diagram for describing that a processor 130 of the apparatus 10 according to the inventive concept calculates coordinate values of a virtual reality-based virtual surgical tool through actual surgical videos.

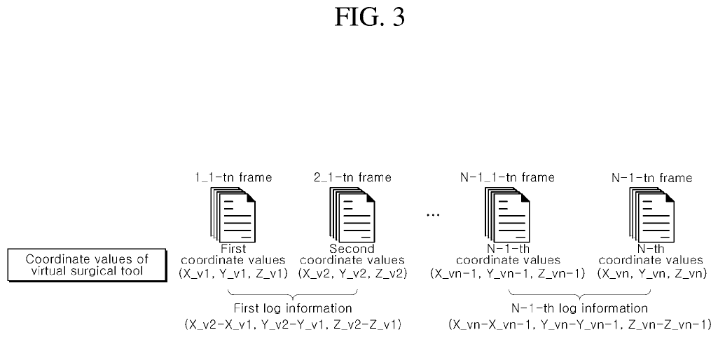

FIG. 3 is a diagram for describing that the processor 130 of the apparatus 10 according to the inventive concept generates log information of a virtual reality-based virtual surgical tool through actual surgical videos.

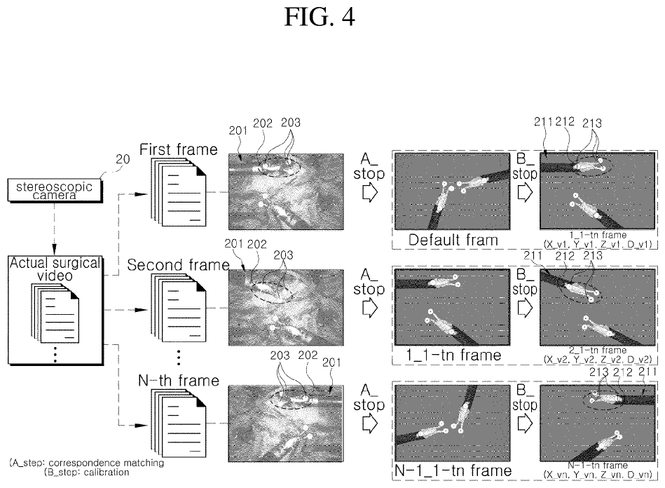

FIG. 4 is a diagram for describing that the processor 130 of the apparatus 10 according to the inventive concept calculates coordinate values of a virtual reality-based virtual surgical tool through actual surgical videos to which depth values are applied.

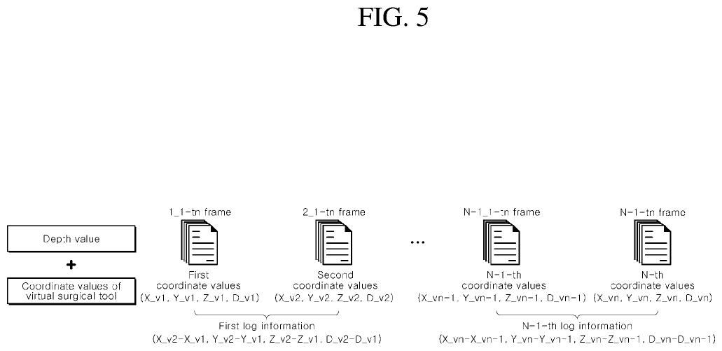

FIG. 5 is a diagram for describing that the processor 130 of the apparatus 10 according to the inventive concept generates log information of a virtual reality-based virtual surgical tool through actual surgical videos to which depth values are applied.

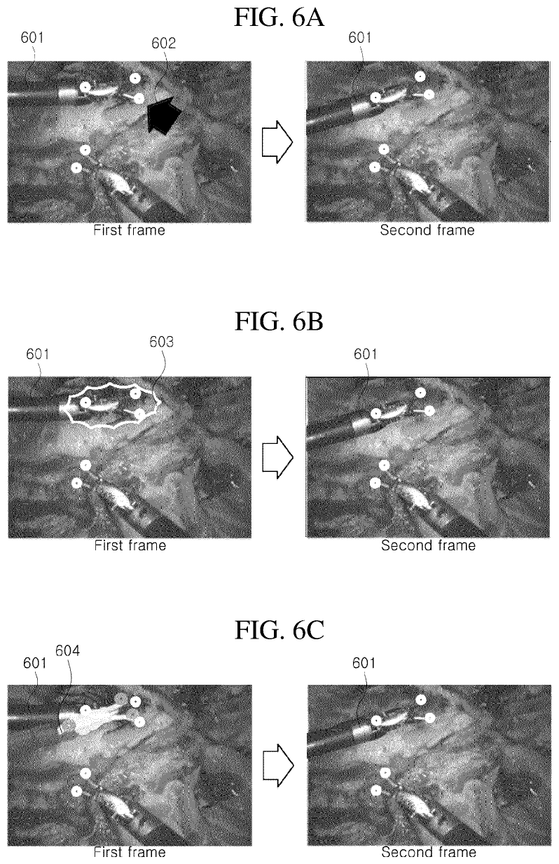

FIGS. 6A to 6C are diagrams for describing that the processor 130 of the apparatus 10 according to the inventive concept displays a visual effect indicating a predicted movement on an actual surgical video.

Hereinafter, the apparatus 10 for providing a virtual reality-based surgical environment according to the inventive concept will be described with reference to FIGS. 1 to 6C. Here, the apparatus 10 may be implemented with a server device as well as a local computing device.

The apparatus 10 may generate a virtual surgical tool identical to an actual surgical tool in an actual surgical video based on virtual reality and determine the movement of the actual surgical tool according to the log information of the virtual surgical tool, thereby accurately identifying the movement of the actual surgical tool.

First, referring to FIG. 1, the apparatus 10 may include a video acquisition unit 110, a memory 120, and a processor 130. Here, the apparatus 10 may include fewer components or more components than the components shown in FIG. 1.

The video acquisition unit 110 may acquire an actual surgical video from an external device (not shown) at a preset period, in real time, or at a point time when a user input is received. Alternatively, the video acquisition unit 110 may acquire the actual surgical video through the memory 120.

Here, the video acquisition unit 110 may include a communication module 111.

The communication module 111 may include one or more modules that enable wireless communication between the apparatus 10 and a wireless communication system or between the apparatus 10 and an external device (not shown). In addition, the communication module 111 may include one or more modules that connect the apparatus 10 to one or more networks.

The memory 120 may store information supporting various functions of the apparatus 10. The memory 120 may store a plurality of application programs (or applications) running on the apparatus 10, and data and instructions for operation of the apparatus 10. At least some of these application programs may be downloaded from an external server (not shown) through wireless communication. In addition, at least some of these application programs may exist for basic functions of the apparatus 10. Meanwhile, the application program may be stored in the memory 120, installed on the apparatus 10, and driven by the processor 130 to perform an operation (or function) of the apparatus 10.

Also, the memory 120 may store a first artificial intelligence model for recognizing at least one actual surgical tool included in the actual surgical video. Here, the first artificial intelligence model may include, but is not limited to, a convolutional neural network (CNN) and a recurrent neural network, and may be formed of neural networks having various structures.

Hereinafter, the Convolutional Neural Network will be referred to as “CNN”, and the Recurrent Neural Network will be referred to as “RNN”.

CNN may be formed in a structure in which a convolution layer that creates a feature map by applying a plurality of filters to regions of an image and a pooling layer that extracts features that are invariant to changes in position or rotation by spatially integrating the feature map are alternately repeated several times. Through this, various levels of features, from low-level of features such as points, lines, and planes to complex and meaningful high-level of features, may be extracted.

The convolutional layer may obtain a feature map by taking a nonlinear activation function on the dot product of a filter and a local receptive field for each patch of an input video. Compared with other network structures, CNNs may have the feature of using filters with sparse connectivity and shared weights. Such a connection structure may reduce the number of parameters to be learned, make learning through the backpropagation algorithm efficient, thus consequently improving prediction performance.

The pooling layer (or sub-sampling layer) may generate a new feature map by using local information of the feature map obtained from the previous convolutional layer. In general, the newly created feature map by the pooling layer may be reduced to a smaller size than the original feature map. Representative pooling methods may include a max pooling method which selects the maximum value of a corresponding region in the feature map, and an average pooling method which obtains the average value of a corresponding region in the feature map. In general, the feature map of the pooling layer may be less affected by the location of arbitrary structures or patterns existing in an input video than the feature map of the previous layer. That is, the pooling layer may enable features that are more robust to local changes such as noise or distortion in the input video or a previous feature map to be extracted, and these features may play an important role in classification performance. Another role of the pooling layer may be to reflect the features of a wider region as it goes to the upper learning layer in the deep structure and to generate features that reflect local features in the lower layer as feature extraction layers are piled up and more abstract features of the entire video as it goes to the upper layer.

As described above, the features finally extracted through repetition of the convolutional layer and the pooling layer may be used for training and prediction for the classification models in such a way that the classification models such as a multi-layer perceptron (MLP) or a support vector machine (SVM) are connected in the form of the fully-connected layer.

RNN is an effective deep learning technique for learning sequence through a structure in which a specific part is repeated and may influence result because a state value of the previous state is input to a next computation.

In addition, the memory 120 may store the at least one actual surgical video acquired through the video acquisition unit 110. Alternatively, the memory 120 may store the at least one actual surgical video in advance. Here, the actual surgical video may be a video in which at least one actual surgical tool included in a surgical video obtained by recording a surgical procedure in an operating room of a hospital or a laboratory environment is captured.

More specifically, the memory 120 may store each of the at least one actual surgery video to be matched with an operation type, an operator name, a hospital name, a patient status, an operation time, an operation environment, and the like.

In addition, the memory 120 may store a three-dimensional depth value to be included in the at least one actual surgical video or separately. More specifically, the memory 120 may store the three-dimensional depth value for the actual surgical object to be included in the at least one actual surgical video captured by a stereoscopic camera 20 or separately, for each frame. Here, the processor 130 may obtain the three-dimensional depth value (three-dimensional depth map) by utilizing multi-view geometry for the actual surgical video taken through the stereoscopic camera 20.

Also, the memory 120 may accumulate and store a plurality of pieces of log information generated according to a difference between a plurality of coordinate values for a position at which the virtual surgical tool is calibrated for each preset frame through the processor 130.

The processor 130 may generally control the overall operation of the apparatus 10 as well as the operation related to the application program. The processor 130 may provide or process information or a function appropriate to a user by processing signals, data, information, and the like, which are input or output through the above-described components, or by executing an application program stored in the memory 120.

In addition, the processor 130 may control at least some of the components described with reference to FIG. 1 in order to execute an application program stored in the memory 120. In addition, the processor 130 may operate at least two or more of the components included in the apparatus 10 in a combination thereof to execute the application program.

Hereinafter, the operation of the processor 130 will be described in detail with reference to FIGS. 2 to 6C.

The processor 130 may recognize at least one actual surgical tool included in an actual surgical video for each preset frame in the actual surgical video based on the first artificial intelligence model (step A).

Alternatively, the processor 130 may recognize at least one actual surgical tool included in an actual surgical video for each preset frame in the actual surgical video based on the first artificial intelligence model (step A). Here, the actual surgical video may be captured through a stereoscopic camera 20 and include a three-dimensional depth value for an actual surgical object for each frame. That is, the three-dimensional depth value for the actual surgical object, such as an actual surgical tool, an actual surgical organ, or a surgeon's hand during an actual operation may be included in the actual surgical video, or may be separately stored to be matched with the actual surgical video in the memory 120.

Here, the processor 130 may recognize at least one actual surgical tool included in the actual surgical video for each frame of the first to N-th frames that are all frames of the actual surgical video. Alternatively, the processor 130 may recognize at least one actual surgical tool included in the actual surgical video for every preset number of frames for the first to N-th frames that are all frames of the actual surgical video.

For example, the processor 130 may recognize two actual surgical tools 201 included in the actual surgical video for each frame of the first to N-th frames in the actual surgical video based on the first artificial intelligence model.

The processor 130 may perform correspondence matching on at least one identical portion of the actual surgical tool and at least one virtual reality-based virtual surgical tool corresponding to the actual surgical tool (step B). Here, each frame of the actual surgical video and each frame generated based on virtual reality may correspond one-to-one.

In more detail, the processor 130 may set at least one portion of the actual surgical tool as a first reference point. Here, the processor 130 may set a region identical to at least one region of the actual surgical tool in the virtual surgical tool as a second reference point by using a semantic correspondence matching technique. Thereafter, the processor 130 may perform the correspondence matching using the first and second reference points. Here, the first and second reference points may mean at least one point.

For example, the processor 130 may perform correspondence matching on identical portion 202 and 212 of the actual surgical tool 201 and at least one of the two virtual reality-based virtual surgical tools 211 for each frame of the first to N-th frames.

In more detail, the processor 130 may set a specific region 202 of the actual surgical tool as the first reference point 203. The processor 130 may set the portion 212 identical to the specific portion 202 of the actual surgical tool 201 in the virtual surgical tool 211 as the second reference point 213 through the semantic correspondence matching technique. The processor 130 may perform the correspondence matching using the first reference point 203 and the second reference point 213.

The processor 130 may perform calibration such that the virtual surgical tool corresponds to the position of the actual surgical tool according to a result of the correspondence matching (step C).

As an example, referring to FIG. 2, the processor 130 may perform calibration such that the virtual surgical tool 211 is positioned as shown in the 1_1-th frame, which had been positioned as shown in a default frame based on virtual reality, according to the result of the correspondence matching performed in the virtual reality-based 1_1-th frame matching a first frame of the actual surgical video. In addition, the processor 130 may perform calibration such that the virtual surgical tool 211 is positioned as shown in the 2_1-th frame, which had been positioned as shown in the 1_1-th frame based on virtual reality, according to the result of the correspondence matching performed in the virtual reality-based 2_1-th frame matching a second frame of the actual surgical video.

As another example, referring to FIG. 4, the processor 130 may render the virtual surgical tool as a three-dimensional object by assigning a corresponding three-dimensional depth value to the calibrated position of the virtual surgical tool. In addition, the processor 130 may calculate a plurality of coordinate values for a position at which the virtual surgical tool rendered as the three-dimensional object is calibrated for each of the preset frames. Here, the processor 130 may have an effect of obtaining a more accurate rendering result when an actual surgical video (e.g., a stereoscope video) having a depth map captured through the stereoscopic camera 20 is utilized.

The processor 130 may calculate the calibrated coordinate values of the virtual surgical tool (step D).

As an example, referring to FIG. 2, the processor 130 may calculate first coordinate values (X_v1, Y_v1, and Z_v1) of the virtual surgical tool in the virtual reality-based 1_1-th frame. In addition, the processor 130 may calculate second coordinate values (X_v2, Y_v2, and Z_v2) of the virtual surgical tool in the virtual reality-based 2_1-th frame. In addition, the processor 130 may calculate N−1-th coordinate values (X_vn−1, Y_vn−1, and Z_vn−1) of the virtual surgical tool in the virtual reality-based N−1_1-th frame. In addition, the processor 130 may calculate N-th coordinate values (X_vn, Y_vn, and Z_vn) of the virtual surgical tool in the virtual reality-based N_1-th frame.

As another example, referring to FIG. 4, the processor 130 may calculate first coordinate values (X_v1, Y_v1, Z_v1, and D_v1) of the virtual surgical tool in the virtual reality-based 1_1-th frame. Also, the processor 130 may calculate second coordinate values (X_v2, Y_v2, Z_v2, and D_v2) of the virtual surgical tool in the virtual reality-based 2_1-th frame. In addition, the processor 130 may calculate N−1-th coordinate values (X_vn−1, Y_vn−1, Z_vn−1, and D_vn−1) of the virtual surgical tool in the virtual reality-based N−1_1-th frame. In addition, the processor 130 may calculate N-th coordinate values (X_vn, Y_vn, Z_vn, and D_vn) of the virtual surgical tool in the virtual reality-based N_1-th frame. Here, the processor 130 may display a virtual surgical tool having a three-dimensional depth value by assigning a depth value when calculating the first to N-th coordinate values of the virtual surgical tool in the virtual reality-based 1_1-th to N_1-th frames. Also, the processor 130 may store coordinate values by including the three-dimensional depth value in the coordinate values corresponding to the calibrated position of the virtual surgical tool after the three-dimensional depth value is assigned.

That is, the processor 130 may calculate a plurality of coordinate values for positions at which the virtual surgical tool is calibrated for each preset frame by repeatedly performing steps A to D.

The processor 130 may generate a plurality of pieces of log information based on a difference between the calculated plurality of coordinate values.

More specifically, whenever the plurality of coordinate values are calculated, the processor 130 may sequentially generate log information about a difference between a previously-calculated coordinate value and a subsequently-calculated coordinate value.

As an example, referring to FIG. 3, the processor 130 may generate first log information (X_v2-X_v1, Y_v2-Y_v1, and Z_v2-Z_v1) which is log information about the difference between first coordinate values (X_v1, Y_v1, and Z_v1) and second coordinate values (X_v2, Y_v2, and Z_v2). In this way, the processor 130 may continue to sequentially generate log information about a difference between the coordinate value of one frame and the coordinate value of a frame immediately after the one frame. That is, the processor 130 may generate N−1-th log information (X_vn-X_vn−1, Y_vn-Y_vn−1, and Z_vn-Z_vn−1) which is log information about a difference between the N-th coordinate values (X_vn, Y_vn, and Z_vn) and the N−1-th coordinate values (X_vn−1, Y_vn−1, and Z_vn−1).

As another example, referring to FIG. 5, the processor 130 may generate first log information (X_v2-X_v1, Y_v2-Y_v1, Z_v2-Z_v1, and D_v2-D_v1) which is log information including a depth value for the difference between the first coordinate values (X_v1, Y_v1, Z_v1, and D_v1) and the second coordinate values (X_v2, Y_v2, Z_v2, and D_v2). In this way, the processor 130 may continue to sequentially generate log information about a difference between the coordinate value of one frame and the coordinate value of a frame immediately after the one frame. That is, the processor 130 may generate N−1-th log information (X_vn-X_vn−1, Y_vn-Y_vn−1, Z_vn-Z_vn−1, and D_vn-D_vn−1) which is log information including a depth value for a difference between the N-th coordinate values (X_vn, Y_vn, Z_vn, and D_vn) and the N−1-th coordinate values (X_vn−1, Y_vn−1, Z_vn−1, and D_vn−1).

The processor 130 may accumulate and store sequentially generated pieces of log information in the memory 120.

In more detail, the processor 130 may accumulate and store currently generated log information only when a difference between the sequentially generated pieces of log information is equal to or greater than a preset difference.

That is, when the difference between the first log information generated while transitioning from the first frame to the second frame and the second log information generated while transitioning from the second frame to the third frame is less than a preset difference, the processor 130 may again store first log information as it is. On the other hand, when seventh log information generated while transitioning from the seventh frame to the eighth frame is equal to or greater than the preset difference, the processor 130 may not accumulate and store the previously generated sixth log information, but accumulate and store the seventh log information.

The processor 130 may predict the movement of the actual surgical tool changed from the current frame to the next frame in the actual surgical video based on the accumulated and stored log information.

The processor 130 may display a visual effect indicating the predicted movement at a position corresponding to the movement on the current frame in the actual surgical video.

More specifically, the processor 130 may display a visual effect indicating the predicted movement on each frame of the stored actual surgery video at a position corresponding to the movement in the next frame of the actual surgical tool.

For example, referring to FIG. 6A, the processor 130 may predict the movement of the actual surgical tool 601 that is changed from the first frame to the second frame in the actual surgical video, and display a first visual effect 602 with an arrow-shaped marker representing the predicted movement, at a position corresponding to the movement in the first frame.

As another example, referring to FIG. 6B, the processor 130 may predict the movement of the actual surgical tool 601 that is changed from the first frame to the second frame in the actual surgical video, and display a first visual effect 603 with a blinking shape representing the predicted movement, at a position corresponding to the movement in the first frame.

As another example, referring to FIG. 6C, the processor 130 may predict the movement of the actual surgical tool 601 that is changed from the first frame to the second frame in the actual surgical video, and display a first visual effect 604 in the form of a semi-transparent actual surgical tool representing the predicted movement, at a position corresponding to the movement in the first frame.

Accordingly, the processor 130 may display the predicted movement of the actual surgical tool 601 on the actual surgical video, thus making it possible to provide a guided surgery, when users practice the surgery while watching the actual surgery video and provide reliable and practical help.

The processor 130 may determine a type of surgery for the actual surgery video on which the log information is generated through the log information, and when the surgery is performed using a same type of robot according to the determined type of surgery, remotely control the robot using the log information.

The processor 130 may perform a surgical analysis on the actual surgical video through the log information. In more detail, the processor 130 may determine that an event has occurred when a difference between previously generated log information and immediately subsequent log information among the log information is equal to or greater than a preset difference. Accordingly, the processor 130 may determine a specific surgical procedure with respect to the actual surgical video.

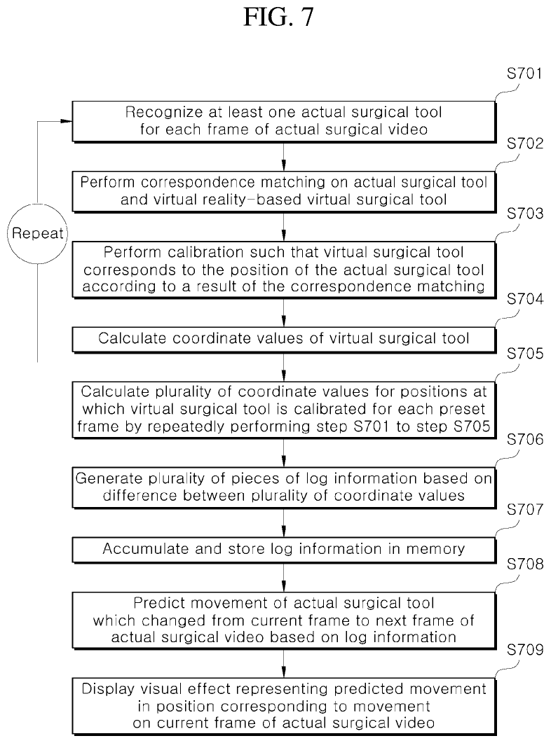

FIG. 7 is a flowchart illustrating a process in which the processor 130 of the apparatus 10 according to the inventive concept provides a virtual reality-based surgical environment.

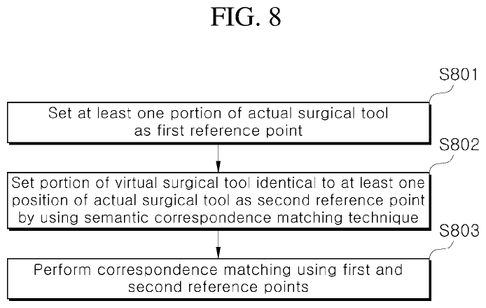

FIG. 8 is a flowchart illustrating a process in which the processor 130 of the apparatus 10 according to the inventive concept performs correspondence matching.

In FIGS. 7 to 8, an operation of the processor 130 may be performed by the apparatus 10.

The processor 130 may recognize at least one actual surgical tool for each frame in an actual surgical video (S701).

Specifically, the processor 130 may recognize at least one actual surgical tool included in an actual surgical video for each preset frame in the actual surgical video based on the first artificial intelligence model.

The processor 130 may perform correspondence matching on the actual surgical tool and a virtual reality-based virtual surgical tool (S702).

Specifically, the processor 130 may perform correspondence matching on at least one identical portion of the actual surgical tool and at least one virtual reality-based virtual surgical tool corresponding to the actual surgical tool.

In step S702, more specifically, referring to FIG. 8, the processor 130 may set at least one portion of the actual surgical tool as a first reference point (S801).

The processor 130 may set a region in the virtual surgical tool identical to at least one region of the actual surgical tool as a second reference point by using a semantic correspondence matching technique (S802).

The processor 130 may perform the correspondence matching using the first and second reference points (S803).

The processor 130 may perform calibration such that the virtual surgical tool corresponds to the position of the actual surgical tool according to a result of the correspondence matching (S703).

The processor 130 may calculate the calibrated coordinate values of the virtual surgical tool (S704).

The processor 130 may calculate a plurality of coordinate values for positions at which the virtual surgical tool is calibrated for each preset frame by repeatedly performing steps S701 to S704 (S705).

Here, when the actual surgical video is captured by the stereoscopic camera 20 and has a three-dimensional depth value, the processor 130 may render the virtual surgical tool as a three-dimensional object by assigning the corresponding three-dimensional depth value to the calibrated position of the virtual surgical tool. In addition, the processor 130 may calculate a plurality of coordinate values for a position at which the virtual surgical tool rendered as the three-dimensional object is calibrated for each of the preset frames.

The processor 130 may generate a plurality of pieces of log information based on a difference between the calculated plurality of coordinate values (S706).

More specifically, whenever the plurality of coordinate values are calculated, the processor 130 may sequentially generate log information about a difference between a previously-calculated coordinate value and a subsequently-calculated coordinate value.

Alternatively, the processor 130 may generate a plurality of pieces of log information to which the three-dimensional depth value is assigned based on a difference between a plurality of coordinate values calculated by including the three-dimensional depth value.

The processor 130 may accumulate and store the sequentially-generated log information in the memory 120 (S707).

In more detail, the processor 130 may accumulate and store currently generated log information only when a difference between the sequentially generated pieces of log information is equal to or greater than a preset difference.

The processor 130 may predict the movement of the actual surgical tool changed from the current frame to the next frame in the actual surgical video based on the accumulated and stored log information (S708).

The processor 130 may display a visual effect representing the predicted movement at a position corresponding to the movement on the current frame in the actual surgical video (S709).

Although it is described with reference to FIGS. 7 and 8 that a plurality of steps are sequentially performed, this is merely illustrative of the technical idea of the embodiment. Those of ordinary skill in the art to which this embodiment belongs may perform various modifications and variations such as changing the order described in FIGS. 7 to 8 or performing one or more of the plurality of steps in parallel without departing from the essential features of the present embodiment, so that the inventive concept is not limited to chronological order in FIGS. 7 to 8.

The method according to the inventive concept described above may be implemented as a program (or application) to be executed in combination with a server, which is hardware, and stored in a medium.

The above-described program may include codes coded in a computer language, such as C, C++, JAVA, or a machine language, which are readable by a processor (CPU) of the computer through a device interface of the computer such that the computer reads the program and executes the methods implemented by the program. The codes may include functional codes associated with a function defining functions necessary to execute the methods or the like, and include control codes associated with an execution procedure necessary for the processor of the computer to execute the functions according to a predetermined procedure. In addition, the codes may further include memory reference codes indicating at which location (address number) of the computer's internal or external memory, additional information or media required for the computer's processor to execute the functions can be referenced. In addition, when the processor of the computer needs to communicate with any other computer or server located remotely to execute the above functions, codes may further include communication-related codes for how to communicate with any other remote computer or server using a communication module of the computer, and what information or media to transmit/receive during communication.

The storage medium refers to a medium that stores data semi-permanently rather than a medium storing data for a very short time, such as a register, a cache, and a memory, and is readable by an apparatus. Specifically, examples of the storage medium may include, but are not limited to, a ROM, a RAM, a CD-ROM, a magnetic tape, a floppy disk, an optical data storage device, and the like. That is, the program may be stored in various recording media on various servers to which the computer can access or various recording media on the computer of a user. The medium may also be distributed to a computer system connected thereto through a network and store computer readable codes in a distributed manner.

The steps of a method or algorithm described in connection with the embodiments of the present disclosure may be implemented directly in hardware, in a software module executed by hardware, or in a combination thereof. The software module may reside in a random access memory (RAM), a read only memory (ROM), an erasable programmable ROM (EPROM), an electrically erasable programmable ROM (EEPROM), a flash memory, a hard disk, a removable disk, a CD-ROM, or in a computer readable recording medium that is well known in the art.

Although embodiments of the present disclosure have been described above with reference to the accompanying drawings, it is understood that those skilled in the art to which the present disclosure pertains may implement the present disclosure in other specific forms without changing the technical spirit or essential features thereof. Therefore, it should be understood that the embodiments described above are illustrative in all respects and not restrictive.

According to the inventive concept, it is possible to generate a virtual surgical tool identical to an actual surgical tool in an actual surgical video based on virtual reality and determine the movement of the actual surgical tool according to the log information of the virtual surgical tool, thereby accurately identifying the movement of the actual surgical tool.

However, the effects of the inventive concept may not be limited to the above-described effects. Although not described herein, other effects to be solved by the inventive concept can be clearly understood by those skilled in the art from the following description.

While the inventive concept has been described with reference to embodiments, it will be apparent to those skilled in the art that various changes and modifications may be made without departing from the spirit and scope of the inventive concept. Therefore, it should be understood that the above embodiments are not limiting, but illustrative.

What is claimed is:

1. A method of providing surgical environment based on a virtual reality in an apparatus, the method comprising:
a) recognizing at least one actual surgical tool included in an actual surgical video for each preset frame in the actual surgical video based on a first artificial intelligence model;
b) performing correspondence matching on at least one identical portion of the actual surgical tool and at least one virtual reality-based virtual surgical tool corresponding to the actual surgical tool;
c) performing calibration such that the virtual surgical tool corresponds to a position of the actual surgical tool according to a result of the correspondence matching;
d) calculating calibrated coordinate values of the virtual surgical tool;
e) calculating a plurality of coordinate values for positions which the virtual surgical tool is calibrated for each preset frame by repeatedly performing steps a) to d); and
f) generating a plurality of pieces of log information based on a difference between the calculated plurality of coordinate values.

2. The method of claim 1, wherein the performing of the correspondence matching includes
setting at least one region of the actual surgical tool as a first reference point;
setting a region identical to the at least one region of the actual surgical tool in the virtual surgical tool as a second reference point by using a semantic correspondence matching technique; and
performing the correspondence matching using the first and second reference points.

3. The method of claim 2, wherein the generating of the log information includes sequentially generating pieces of log information about a difference between a previously-calculated coordinate value and a subsequently-calculated coordinate value whenever the plurality of coordinate values are calculated.

4. The method of claim 3, further comprising:
accumulating and storing the sequentially generated pieces of log information.

5. The method of claim 4, wherein the accumulating and storing of the sequentially generated pieces of log information includes
accumulating and storing currently generated log information only when a difference between the sequentially generated pieces of log information is equal to or greater than a preset difference.

6. The method of claim 4, further comprising:
predicting a movement of the actual surgical tool changed from a current frame to a next frame in the actual surgical video based on the accumulated and stored log information; and
displaying a visual effect representing the predicted movement at a position corresponding to the movement on the current frame in the actual surgical video.

7. The method of claim 1, wherein the actual surgical video is taken through a stereoscopic camera and includes a three-dimensional depth value for an actual surgical object for each frame, and
wherein the performing of the calibration includes rendering the virtual surgical tool as a three-dimensional object by assigning a corresponding three-dimensional depth value to the calibrated position of the virtual surgical tool.

8. The method of claim 7, wherein the calculating of the coordinate values includes
including the three-dimensional depth value in coordinate values corresponding to the calibrated position of the virtual surgical tool after the three-dimensional depth value is assigned.

9. The method of claim 8, wherein the generating of the log information includes
generating a plurality of pieces of log information to which the three-dimensional depth value is assigned based on a difference between a plurality of coordinate values calculated by including the three-dimensional depth value.

10. An apparatus for providing surgical environment based on a virtual reality, comprising:
a video acquisition unit configured to acquire an actual surgical video;
a memory; and
a processor configured to:
perform a first process of recognizing at least one actual surgical tool included in an actual surgical video for each preset frame in the actual surgical video based on a first artificial intelligence model,
perform a second process of performing correspondence matching on at least one identical portion of the actual surgical tool and at least one virtual reality-based virtual surgical tool corresponding to the actual surgical tool;
perform a third process of performing calibration such that the virtual surgical tool corresponds to a position of the actual surgical tool according to a result of the correspondence matching;
perform a fourth process of calculating calibrated coordinate values of the virtual surgical tool;
perform a fifth process of calculating a plurality of coordinate values for positions which the virtual surgical tool is calibrated for each preset frame by repeatedly performing the first to fourth process; and
perform a sixth process of generating a plurality of pieces of log information based on a difference between the calculated plurality of coordinate values.

11. The apparatus of claim 10, wherein the processor is configured to:
set at least one region of the actual surgical tool as a first reference point when performing the correspondence matching,
set a region identical to the at least one region of the actual surgical tool in the virtual surgical tool as a second reference point by using a semantic correspondence matching technique, and
perform the correspondence matching using the first and second reference points.

12. The apparatus of claim 11, wherein the processor is configured to sequentially generate log information about a difference between a previously-calculated coordinate value and a subsequently-calculated coordinate value whenever the log information is generated.

13. The apparatus of claim 12, wherein the processor is configured to:
accumulate and store the sequentially generated log information,
wherein the processor is configured to:
accumulate and store the currently generated log information only when a difference between the sequentially generated log information is equal to or greater than a preset difference when the log information is accumulated and stored.

14. The apparatus of claim 13, wherein the processor is configured to:
predict a movement of the actual surgical tool changed from a current frame to a next frame in the actual surgical video based on the accumulated and stored log information; and
display a visual effect indicating the predicted movement at a position corresponding to the movement on the current frame in the actual surgical video.

15. The apparatus of claim 11, wherein the actual surgical video is taken through a stereoscopic camera and includes a three-dimensional depth value for an actual surgical object for each frame, and
wherein the processor is configured to render the virtual surgical tool as a three-dimensional object by assigning a corresponding three-dimensional depth value to the calibrated position of the virtual surgical tool when performing calibration on the virtual surgical tool.

16. The apparatus of claim 15, wherein the processor is configured to:
include the three-dimensional depth value in coordinate values corresponding to the calibrated position of the virtual surgical tool after the three-dimensional depth value is assigned when the coordinate values are calculated, and
generate a plurality of pieces of log information to which the three-dimensional depth value is assigned based on a difference between a plurality of coordinate values calculated by including the three-dimensional depth value when the log information is generated.

17. A computer program stored in a computer-readable recording medium to execute the method for providing a virtual reality-based surgical environment in the apparatus in cooperation with a computer in claim 1.
